(19) United States
(12) Patent Application Publication
(10) Pub. No.: US 2004/0063099 A1
(43) Pub. Date: Apr. 1, 2004

(54) Methods, systems and software for biological analysis

(75) Inventor: Jacques Retieff, Sunnyvale, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/256,938

(22) Filed: Sep. 27, 2002

(51) Int. Cl. C12Q 1/68; G06F 19/00; G01N 33/48; G01N 33/50

(52) U.S. Cl. 435/6; 702/20

(57) ABSTRACT

Methods, computer software and systems are provided for biological data analysis. In one embodiment, significantly changed measurements are mapped to a biological function, such as a GO (Gene Ontology) annotation, category or biological pathway.

### BACKGROUND OF THE INVENTION

This invention is related to bioinformatics and biological data analysis and visualization.

Many biological functions are carried out by regulating the expression levels of various genes, either through changes in the copy number of the genetic DNA, through changes in levels of transcription (e.g. through control of initiation, provision of RNA precursors, RNA processing, etc.) of particular genes, or through changes in protein synthesis. For example, control of the cell cycle and cell differentiation, as well as diseases, are characterized by the variations in the transcription levels of a group of genes.

Recently, massive parallel gene expression monitoring methods have been developed to monitor the expression of a large number of genes using nucleic acid array technology which was described in detail in, for example, U.S. Pat. No. 5,871,928; de Saizieu, et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3.

Massive parallel gene expression monitoring experiments generate unprecedented amounts of information. Effective analysis of the large amount of data may lead to the development of new drugs and new diagnostic tools. Therefore, there is a great demand in the art for methods for organizing, accessing and analyzing the vast amount of information collected using massive parallel gene expression monitoring methods.

### SUMMARY OF THE INVENTION

In one aspect of the invention, methods, systems and computer software products are provided for conducting biological analysis. The methods, systems and computer software products are particularly suitable for analyzing gene expression data preferably obtained using microarray technology. However, the methods, systems and computer software products are also suitable for analyzing other types of biological data, such as protein profile data.

Biological measurements are analyzed using standard statistical methods by, e.g., calculating a statistically significant cut-off, such as a p-value from a t-test or an ANOVA model, for measurements that have changed between experimental conditions (FIG. 1, 101). The statistical tests can be views as a filter to detect measurements that are significantly altered under specific experimental conditions. Typically a very conservative cutoff, such as a Bonferroni correction may be used to reduce false positives. This has the effect of decreasing the sensitivity of the experiment. Because a second, complimentary filter (102) to reduce false positives can be used, this primary filter may be relaxed to improve sensitivity.

In some embodiments, all significantly changed measurements are mapped to a biological function, such as a GO (Gene Ontology) annotation, category or biological pathway. By setting a requirement that a certain number of measurements (e.g., greater than 2, 3, 4, 5, 6, 7, 10) must belong to a particular annotation, false positives can be greatly reduced. The output (103) is the biological function, pathway, or GO annotation that is perturbed under specific experimental conditions.

The filters used in the statistical analysis step and mapping step are based on different models and are therefore complimentary. For example if a false positive slips through the p-value cutoff, it will probably belong to a random annotation and will be filtered out by the requirement that a certain number, e.g., 4, measurements must belong to the same category. By using two complimentary filters, both high sensitivity and specificity may be achieved.

The exact values for the filter values may be determined independently, usually empirically. Determining the filter values empirically are well within the skills of one of ordinary skill in the art.

The biological information used in the mapping and filtering has an inherent structure. For example, some biological pathways have been studied more extensively than others so more annotations will exist for them. Also, some pathways are inherently more complex and contain more members than others. To ensure the effectiveness of the filter, it may be desirable to use a fraction or percentage of known annotations instead of an absolute number. The ability of this filter to reduce a set of random values will be an indication of its effectiveness.

In some embodiments, the direction of change is not specified. Rather, the data are mapped in terms of perturbing a pathway and not simply as up- or down-regulating the pathway. Many of the significant pathways, such as apoptosis, contain genes that are both up and down regulated—very probable in a well-regulated system.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing one exemplary embodiment of the computerized process for analyzing biological data.

FIG. 2 is a schematic showing one exemplary process for analyzing gene expression data.

FIG. 3 is a schematic showing the exemplary structure of a computer software product for data analysis.

FIG. 4 shows the mapping of all GO biological processes mapped to GeneChip® HG-U133A probe array.

FIG. 5 shows upregulated genes in cells treated with 1,5-Isoquinolinediol, a selective PARP (Poly[ADP-ribose] polymerase inhibitor.

FIG. 6 shows downregulated genes in cells treated with 1,5-Isoquinolinediol, a selective PARP (Poly[ADP-ribose] polymerase inhibitor.

FIG. 7 shows perturbed biological processes when cells are treated with 1,5-Isoquinolinediol, a selective PARP (Poly[ADP-ribose] polymerase inhibitor

### DETAILED DESCRIPTION

Reference will now be made in detail to the exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affyinetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S Pat. No. 6,300,070 and U.S. patent application 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent applications Ser. Nos. 10/063,559, 60/349,546, 60/376,003, 60/394,574, 60/403,381.

Definitions

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point^{TM} fro the specific sequence at s defined ionic strength and pH. The Tm is the temperature (under defined ionic strength, pH and nucleic acid composition) at which 50% of the probes complementary to the target sequence hybridize to the target sequence at equilibrium. Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

In one aspect of the invention, methods, systems and computer software products are provided for conducting biological analysis. The methods, systems and computer software products are particularly suitable for analyzing gene expression data preferably obtained using microarray technology. However, the methods, systems and computer software products are also suitable for analyzing other types of biological data, such as protein profile data.

Biological state can be affected by a numerous factors such as drug treatment, physiological changes, toxicological responses, etc. The biological state of a biological sample (such as a cell, a biopsy tissue sample, serum sample, etc.) can be represented by a number of biological variables. As used herein, the term biological variables are biological measurements or derived from biological measurements. For example, a biological variable can be the expression value of a gene, an index reflecting the expression of a group of genes, the activity of a protein, the concentration of a biological molecule, the conformation of a biological molecule, etc. A collection of the values of biological variables is generally referred to as the “profile” of the biological state of a sample.

Two or more biological states are typically compared by examining the profiles to discover changed biological variables. For example, cells may be treated with a drug and the expression of genes in treated and untreated cells can be compared to detect genes whose expression is altered.

It is well know to one of the skill in the art that statistical analysis can be used to detect any changes in biological variables. Experimental design and statistical analysis methods are the subject of numerous books including, e.g., Abrahamse, A. 1969, The Power of Some Tests in the General Linear Model University of Rotterdam; Aczel, A. 1995 Statistics: Concepts and Applications Richard D. Irwin Inc.; Agresti, A. 1990 Categorical Data Analysis John Wiley and Sons, New York; Aickin, M. 1983 Linear Statistical Analysis of Discrete Data Wiley, New York; Aitchison, J. 1997 Statistical Concepts and Applications in Medicine Chapman and Hall ; Anderson, A. 1989 Interpreting Data: A First Course in Statistics Chapman and Hall/CRC; Anderson, T. 1986 The Statistical Analysis of Data (Second Edition) Scientific Press; Anderson, T. & Finn, J. 1996 The New Statistical Analysis of Data Springer, New York; Anderson, V. L. & McLean, R. A. 1974 Design of Experiments: A Realistic Approach Marcel Dekker, New York; Backhouse, J. 1967 Statistics: An Introduction to Tests of Significance Longmans, London; Bailey, N. T. J. 1981 Statistical Methods in Biology (Second Edition) Hodder and Stoughton, London; Bechhofer, R., Santner, T., & Goldsman, D. 1995 Design and Analysis of Experiments for Statistical Selection, Screening, and Multiple Comparisons Wiley, N.Y.; Behnen, K. & Neuhaus, G. 1989 Rank Tests with Estimated Scores and Their Application B. G. Tuebner, Stuttgart; Brandt, S. 1999 Data Analysis Statistical and Computational Methods for Scientists and Engineers (with CD-ROM) New York, Springer; Campbell, R. 1989 Statistics for Biologists Cambridge University Press; Dean, A. & Voss, D. 1999 Design and Analysis of Experiments Springer, New York; Federer, W. 1955 Experimental Design, Theory and Application Macmillan, New York; Garcia-Diaz, A. & Phillips, D. 1995 Principles of Experimental Design and Analysis Chapman & Hall, London; Harlow, L., Mulaik, S. & Steiger, J. 1997 What if There Were No Significance Tests? Lawrence Erlbaum Associates, Publishers; Snedecor, G. W. & Cochran, W. G. 1980 Statistical Methods (Seventh Edition) Iowa State University Press, Iowa; Yandell, B. 1997 Practical Data Analysis for Designed Experiments CRC Press; Yates, F. 1970 Experimental Design: Selected Papers of Frank Yates Griffin, London ; Zar, J. 1999 Biostatistical Analysis Prentice-Hall, Engelwood Cliffs; Zolman, J. F. 1993 Biostatistics. Experimental Design and Statistical Inference Oxford University Press, Oxford, all incorporated herein by reference. Computer algorithms, software and source code for carrying out various statistical analysis are widely available.

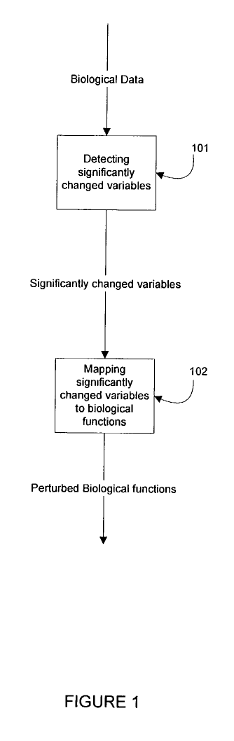

In one aspect of the invention, biological measurements are analyzed using standard statistical methods by, e.g., calculating a statistically significant cut-off, such as a p-value from a t-test, an ANOVA or a nonparametric test, for measurements that have changed between experimental conditions (FIG. 1, 101). The statistical tests can be views as a filter to detect measurements that are significantly altered under specific experimental conditions. Typically a very conservative cutoff, such as a Bonferroni correction may be used to reduce false positives. This has the effect of decreasing the sensitivity of the experiment. Because a second, complimentary filter (102) to reduce false positives can be used, this primary filter may be relaxed to improve sensitivity.

In some embodiments, significantly changed biological variables are mapped to a biological function (FIG. 1, 102), such as a GO (Gene Ontology) annotation, category or biological pathway. By setting a requirement that a certain number of measurements (e.g., greater than 2, 3, 4, 5, 6, 7, 10) must belong to a particular annotation, false positives can be greatly reduced.

Biological functions, as used herein, refers to classifications of any biological functions, processes, cellular components, characteristics, pathways, etc. Examples of biological functions include, e.g., “cell growth and maintenance,” or “signal transduction”, “protein biosynthesis”, “ribonucleoprotein”, etc.

In some embodiments, the classification used by the Gene Ontology Consortium (www. geneontology.com) may be used.

Mapping of biological variables (such as expression of genes) to biological functions can be performance using, for example, Genemapp or Gene Ontology.

The filters used in the statistical analysis step 101 and mapping step 102 are based on different models and are therefore complimentary. For example if a false positive slips through the p-value cutoff, it will probably belong to a random annotation and will be filtered out by the requirement that a certain number, e.g., 4, measurements must belong to the same category. By using two complimentary filters, both high sensitivity and specificity may be achieved.

The exact values for the filter values will need to be determined independently, usually empirically. Determining the filter values empirically are well within the skills of one of ordinary skill in the art.

The biological information used in the mapping and filtering has an inherent structure. For example, some biological pathways have been studied more extensively than others so more annotations will exist for them. Also, some pathways are inherently more complex and contain more members than others. To ensure the effectiveness of the filter, it may be desirable to use a fraction or percentage of known annotations instead of an absolute number. The ability of this filter to reduce a set of random values will be an indication of its effectiveness.

In some embodiments, the direction of change is not specified. Rather, the data are mapped in terms of perturbing a pathway and not simply as up- or down-regulating the pathway. Many of the significant pathways, such as apoptosis, contain genes that are both up and down regulated—very probable in a well-regulated system.

An additional benefit from this approach is that the biological data-interpretation is simplified.

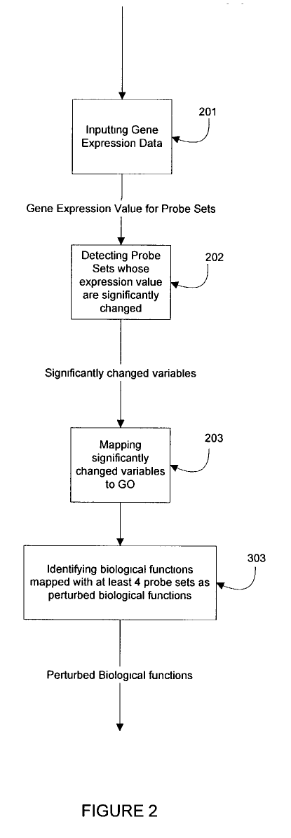

FIG. 2 shows an exemplary process for analyzing gene expression data. Gene expression data are inputted (201). The expression data reflects at least two biological states so that the two states can be compared to find genes whose expression are significantly changed. If the gene expression data are generated using probe sets (e.g., GeneChip® probe arrays, Affymetrix, Santa Clara, Calif., USA). Each value may be the expression value for a probe set. A statistical test, such as ANOVA, is then performed on the data to determine which genes whose expression are significantly changes (as used herein, the term “significant” is intended to mean statistically significant). One of skill in the art would appreciate that this invention is not limited to any specific statistical test or criteria for statistical significance. Typically, a p value smaller than 0.01, 0.05 or 0.10 would indicate a statistical significance.

In some embodiments, the statistical analysis used in this step is a low stringency one. Sometimes, the criteria for statistical significance may be relaxed. Low stringency tests are employed because additional filtering steps are employed for data analysis.

Once the genes whose expression is significantly changed are identified, they are mapped to biological functions according to, e.g., Gene Ontology annotation (203). A biological function that has at least 2, 4, 6, or 8 variables mapped may be identified as a perturbed biological function (303).

Methods useful for identifying perturbed biological functions have extensive applications in, pharmacology, drug discovery, target validation, toxicology, etc. For example, the method can be used to identify targeted biological function of a drug.

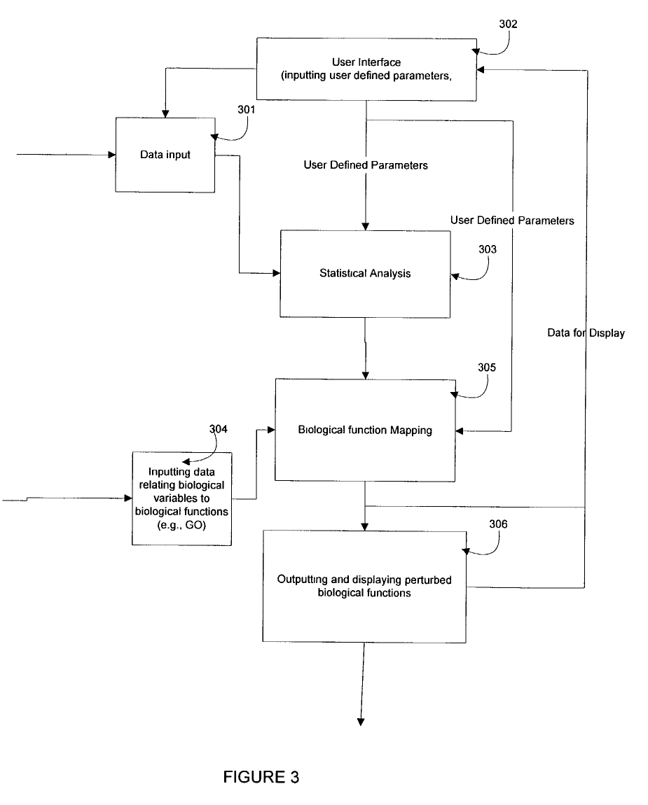

In another aspect of the invention, computer software products are provided for analyzing biological data to identify perturbed biological functions. FIG. 3 is a schematic showing the architecture of one such software product. One of skill in the art would appreciate that this invention is not limited by any particular software architecture. In this exemplary architecture, the software has a data input module (301) which control the input of biological data. The data input module (301) also interacts with a user interface (302). In some embodiments, the software receives input from a user via the user interface for location of the data, for example. User defined parameters may also be received via the user interface. The user defined parameters may include selection of statistical protocols, parameters for statistical analysis, etc. The software may also include a statistical analysis module (303) and a biological function mapping module (305). Optionally, there may be an inputting module to input data relating biological variables to biological functions (305). The data may be from GO consortium annotation. An outputting module (306) can be used to output analysis result. The output may be sent to a computer file, a printout or send to the user interface (302) for display.

The computer software product of the invention may be executed in a single computer or over a network, such as a local area network or a wide area network (e.g., the internet). In a particularly preferred embodiment, the software is executed in an application server for a web server. A user can remotely conduct all the analysis.

A software product typically include a computer readable medium, such as CD-ROM or a DVD Rom disk. Software codes that execute the method steps of the invention are stored in the computer readable medium. Software of the invention can be written in any suitable language including C/C++, Java, C#. Basic, Fortran, Perl, etc.

In yet another aspect of the invention, systems for analyzing biological data are provided. In some embodiments, the system include a central processing unit (CPU) and coupled with the CPU is a memory unit. The system executes the methods steps of the invention.

### EXAMPLES

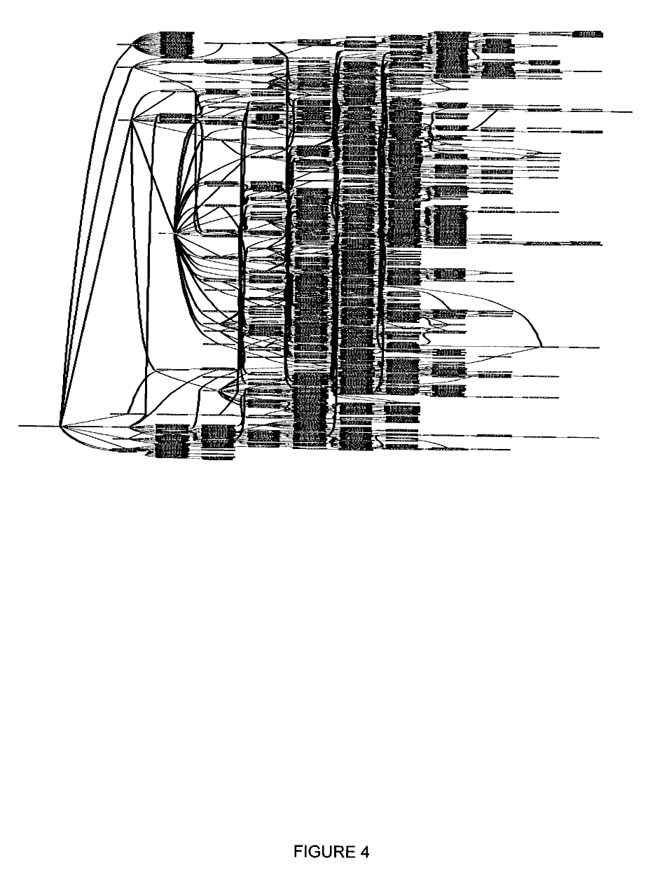

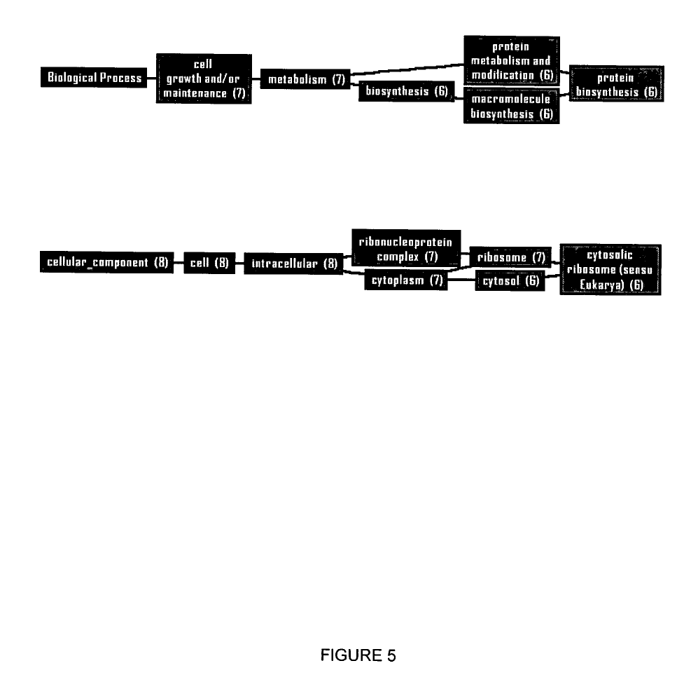

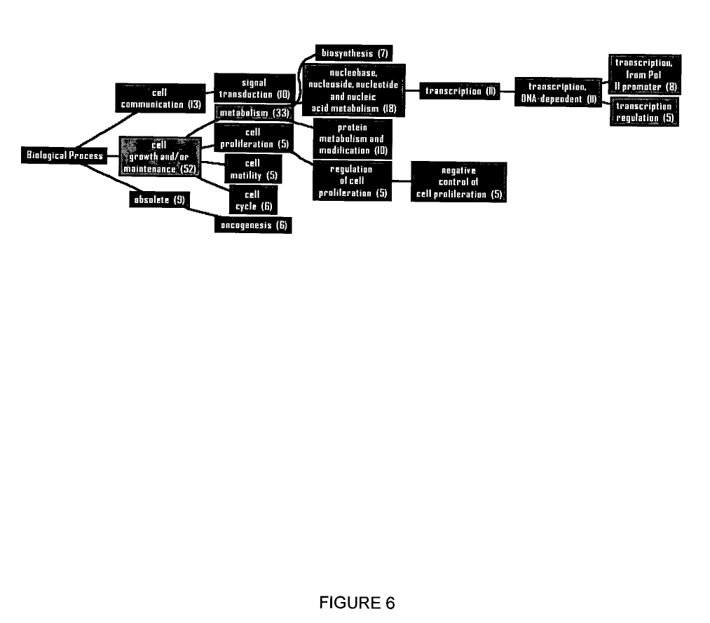

Various embodiments of the invention were employed to analyze gene expression data. FIG. 4 shows the mapping of all genes of a gene expression probe array (GeneChip® Hu133 probe array) to GO annotation. Because of the large number of genes detectable by this array, the mapping is difficult to analyze. FIG. 5 shows the mapping of significantly upregulated genes after the treatment of 1,5-Isoquinolinediol, a selective PARP (Poly[ADP-ribose] polymerase inhibitor. FIG. 6 shows down regulation after similar treatment. Several biological processes such as Cell Growth, metabolism, biosynthesis, protein metabolism, are affected by the treatment.

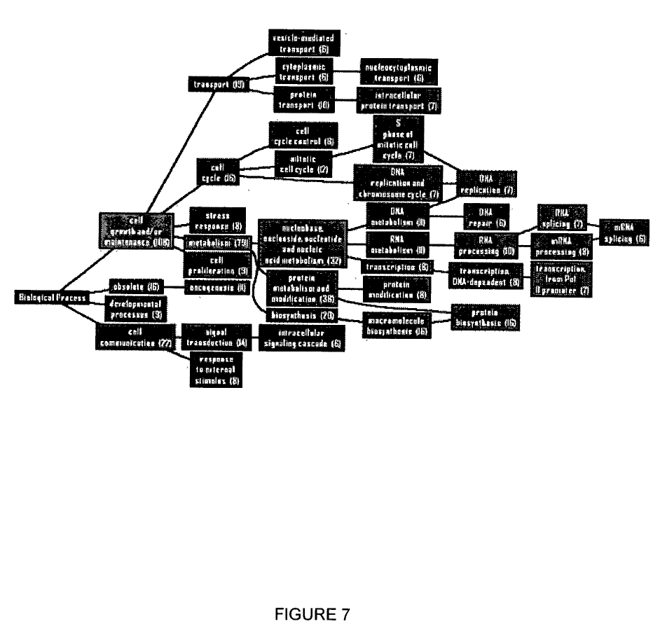

FIG. 7 shows the biological processes affect by PARP treatment. Significantly upregulated and downregulated genes were mapped to biological functions.

The present inventions provide methods and computer software products for analyzing biological data. It is to be understood that the above description is intended to be illustrative and not restrictive. Many variations of the invention will be apparent to those of skill in the art upon reviewing the above description. The scope of the invention should, therefore, be determined not with reference to the above description, but should instead be determined with reference to the appended claims, along with the full scope of equivalents to which such claims are entitled.

All cited references, including patent and non-patent literature, are incorporated herewith by reference in their entireties for all purposes.

What is claimed is:

1. A computerized method for biological data analysis comprising
analyzing data representing at least two biological states to detect significantly changed biological variables;
mapping said significantly changed biological variables to at least one biological function; and
identifying the biological function that is mapped with a minimal number of variables.

2. The method of claim 1 wherein the step of analyzing biological variables comprising performing a statistical comparison to detect said significantly changed biological variables.

3. The method of claim 2 wherein said statistical analysis is a T-test.

4. The method of claim 2 wherein said statistical analysis is analysis of variance.

5. The method of claim 2 wherein said biological function is a Gene Ontology Consortium annotation.

6. The method of claim 2 wherein said biological function is a biological pathway.

7. The method of claim 2 wherein said minimal number of variables is 2.

8. The method of claim 2 wherein said minimal number of variables is 4.

9. The method of claim 2 wherein said biological states are control and treatment.

10. The method of claim 2 wherein each of said biological variables is an expression value of a gene.

11. The method of claim 10 wherein said biological variables represent at least 500 genes.

12. The method of claim 2 wherein said significantly changed variables are significantly increased variables.

13. The method of claim 2 wherein said significantly changed variables are significantly decreased variables.

14. The method of claim 2 wherein said significantly changed variables are significantly increased or decreased variables.

15. A system for managing probe array design data comprising:
a processor; and
a memory coupled with the processor, the memory storing a plurality machine instructions that cause the processor to perform logical steps, wherein the logical steps comprise:
analyzing data representing at least two biological states to detect significantly changed biological variables;
mapping said significantly changed biological variables to at least one biological function; and
identifying the biological function that is mapped with a minimal number of variables.

16. The system of claim 15 wherein the step of analyzing biological variables comprising performing a statistical comparison to detect said significantly changed biological variables.

17. The system of claim 16 wherein said statistical analysis is a T-test.

18. The system of claim 16 wherein said statistical analysis is analysis of variance.

19. The system of claim 16 wherein said biological function is a Gene Ontology Consortium annotation.

20. The system of claim 16 wherein said biological function is a biological pathway.

21. The system of claim 16 wherein said minimal number of variables is 2.

22. The system of claim 16 wherein said minimal number of variables is 4.

23. The system of claim 2 wherein said biological states are control and treatment.

24. The system of claim 16 wherein each of said biological variables is an expression value of a gene.

25. The system of claim 24 wherein said biological variables represent at least 500 genes.

26. The system of claim 16 wherein said significantly changed variables are significantly increased variables.

27. The system of claim 16 wherein said significantly changed variables are significantly decreased variables.

28. The system of claim 16 wherein said significantly changed variables are significantly increased or decreased variables.

29. A computer readable medium comprising computer-executable instructions for performing the methods comprising:
analyzing data representing at least two biological states to detect significantly changed biological variables;
mapping said significantly changed biological variables to at least one biological function; and
identifying the biological function that is mapped with a minimal number of variables.

30. The computer readable medium of claim 29 wherein the step of analyzing biological variables comprising performing a statistical comparison to detect said significantly changed biological variables.

31. The computer readable medium of claim 30 wherein said statistical analysis is a T-test.

32. The computer readable medium of claim 30 wherein said statistical analysis is analysis of variance.

33. The computer readable medium of claim 30 wherein said biological function is a Gene Ontology Consortium annotation.

34. The computer readable medium of claim 30 wherein said biological function is a biological pathway.

35. The computer readable medium of claim 30 wherein said minimal number of variables is 2.

36. The computer readable medium of claim 30 wherein said minimal number of variables is 4.

37. The computer readable medium of claim 30 wherein said biological states are control and treatment.

38. The computer readable medium of claim 30 wherein each of said biological variables is an expression value of a gene.

39. The computer readable medium of claim 38 wherein said biological variables represent at least 500 genes.

40. The computer readable medium of claim 30 wherein said significantly changed variables are significantly increased variables.

41. The computer readable medium of claim 30 wherein said significantly changed variables are significantly decreased variables.

42. The computer readable medium of claim 30 wherein said significantly changed variables are significantly increased or decreased variables.
